(19) United States
(12) Patent Application Publication
(10) Pub. No.: US 2011/0295219 A1
(43) Pub. Date: Dec. 1, 2011

(54) FINGER SHEATH

(76) Inventor: Heng-Jui HSU, Taipei (TW)

(21) Appl. No.: 12/786,445

(22) Filed: May 25, 2010

(51) Int. Cl. A61M 35/00; A45D 40/26

(52) U.S. Cl. 604/289; 132/320

(57) ABSTRACT

A multi-functional finger sheath for cleansing, skin care, scrubbing, smearing, massage, and medical includes at least one opening sized for the insertion of a single or a plurality of finger, and having a length in a range from fingertip to the first knuckle of the finger or from the fingertip to the palm. Said finger sheath is made of an absorber which is a material with water and oil absorbing properties, allowing water-, oil-, milk-, cream- or powered-based materials attached thereon.

### FIELD OF THE INVENTION

The present invention relates to as the multi-functional finger sheaths used for cleansing, skin care, massage and medical use.

### DESCRIPTION OF PRIOR ART

Cotton is an indispensable material used by women in skin care, make-up removal, defoliation, or as a mask. It is commonly used with toner, milk, or even essence. Most cotton pads are square-cut, requiring either the index and middle fingers or middle and ring fingers to hold the pad while wiping. However, the pads often slip away when not properly handled. Furthermore, it is difficult to apply the right amount of force when scrubbing or rubbing for an extended period, resulting in pain, discomfort, and severe muscular inflammation in the fingers.

Traditional cotton pads are square-shaped, and are often difficult to handle and use for other purposes. With modification, the pads will offer a wider range of consumer applications.

### SUMMARY OF THE INVENTION

To solve afore-mentioned drawbacks, the present invention discloses a finger sheath (10) which covers at least one finger for cleansing, skin care, scrubbing, smearing, massage, and medical use is characterized in that said finger sheath (10) having at least one opening (11) to allow the insertion of a single or a plurality of finger with a length in a range from the fingertip to a first knuckle of the finger or from the fingertip to a palm; said sheath (10) is made of material with water absorbing and oil absorbing properties.

Said skin-friendly finger sheath (10) can be a pouched sheath (10A) with at least one opening (11) for finger insertion, while the other end is sealed. Both faces of the sheath (10A) may be used.

Said finger sheath (10) can also be made as a tubular sheath (10B) having opening (11) sized (11) for the insertion of fingers, and both sides thereof may be used.

Said finger sheath (10) can also be two symmetrical fabrics (13, 14) superimposed upon each other, allowing an opening (11) and three other ends sealed.

Said finger sheath (10) is characterized in that a band (10C) having both ends thereof attach respectively to a hook (15) and a loop portion (16) of a hook and loop fastener for adjusting purposes.

Said finger sheath (10) is characterized in that both ends of a bandage (17) are attached onto a plate-like draper (10D). Fingers may be inserted into it with palms-side lying on the flat draper. The bandage (17) may be either a ribbon or an elastic drawstring.

Said finger sheath (10) is characterized in that two lateral sides of plate-like draper (10D) are attached respectively to an end of stripes (18, 19). Another end of stripe (18) attaches to a hook portion (20) of a hook and loop fastener and another end of stripe (19) attaches to a loop portion (21) of said hook and loop fastener. The stripes (18, 19) may be either ribbons or elastic drawstrings.

Said finger sheath (10) is characterized in that an opening (11) is covered with a circular band (22) with both a flexible and contracting edge.

Said finger sheath (10) is characterized in that the surface thereof is coated with a water-soluble layer (23) of liquid, milk, cream, gel, or powder.

Said finger sheath (10) is characterized in that the material for manufacturing said finger sheath (10) is selected from a cotton layer (101), a non-woven fabric layer (102), a fabric having cotton (101) as an inner layer and a non-woven fabric as an outer layer (102).

Said finger sheath (10) is characterized in that the absorber surface may be made of fiber, porous fiber, or a mixture of both.

Said finger sheath (10) is characterized in that a spacer (24) and a separator (25) can be provided therein for spacing the fingers.

### Comparison with Prior Art

As an advantage, the finger sheath of the present invention may be used to replace traditional cotton pads. With the help of these sheaths, less strength is required when rubbing against any surface. Furthermore, the sheath (10) will not slip away from the fingers so easily.

The finger sheath (10) absorbs water and oil-base materials. Different materials are used for different purposes. Other than using it for applying make-up, cosmetics removal, and skin care, it may also be used for shoe-cleaning, massage, medical application, and many other uses.

To ensure safety and hygiene, the finger sheath (10) can also be made as disposable.

The finger sheath (10) can be coupled with a stretchable band (22) to provide better grip, preventing it from slipping off the fingers easily. Furthermore, the inner structure is constructed with either a spacer (24) or a separator (25) to separate the fingers, ensuring better protection of fingers and optimal efficiency of use.

Moreover, the surface of finger sheath (10) may be coated with a water-soluble layer (23) of liquid-, milk-, cream-, gel-, or powder-based material. The finger sheath (10) is sealed in vacuum bag and should be opened only before use. The layer (23) will dissolve when it is mixed with water. In this purpose, additional liquid-, milk-, cream-, gel-, or powder-based cleansers are not required.

### BRIEF DESCRIPTION OF DRAWINGS

FIG. 1 is a cutaway view of the first embodiment of the present invention.

FIG. 2 illustrates four alternative shapes for the first embodiments.

FIG. 3 demonstrates five applicable methods of the first embodiment.

FIG. 4 is a sectional view showing three possible structures for the absorbing material.

FIG. 5 shows two perspective diagrams of the first embodiment which can separate each finger.

FIG. 6 is a side section view of the present invention.

FIG. 7 shows FIG. 6 having a water-soluble layer coupled thereon.

FIG. 8 is a schematic drawing demonstrating the use of the first embodiment.

FIG. 9 is a schematic view showing the first embodiment for skin cleaning.

FIG. 10 is cutaway view of the second embodiment of the present invention.

FIG. 11 is a side section view of the second embodiment.

FIG. 12 is a side section of an alternative version of the second embodiment.

FIG. 13 is a perspective view of the second embodiment applied with spacers.

FIG. 14 is a perspective view of the third embodiment of the present invention.

FIG. 15 is a view showing the deployment of FIG. 14.

FIG. 16 is a plan view illustrating an alternative shape of the third embodiment.

FIG. 17 is a schematic drawing demonstrating the use of the third embodiment.

FIG. 18 is a perspective view of the forth embodiment of the present invention.

FIG. 19 is a schematic drawing demonstrating the use of the forth embodiment.

FIG. 20 is a perspective view of the fifth embodiment of the present invention.

FIG. 21 is a schematic drawing demonstrating the use of the fifth embodiment.

### DETAIL DESCRIPTION OF PREFERRED EMBODIMENTS

The preferred embodiments of the present invention are described in detail according to the appended drawings hereinafter.

### First Embodiment

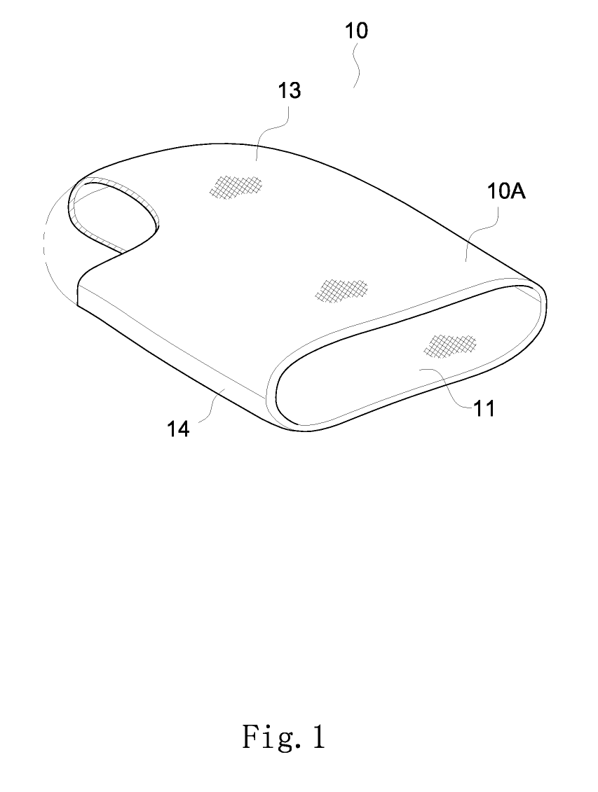

FIG. 1 shows a skin-friendly finger sheath (10), which can be utilized as a tool for cleansing, skin care, scrubbing, and massaging. It can absorb water and oil-based materials, and allow a single or a plurality fingers to be fitted in. The first embodiment of the present invention discloses that said finger sheath (10) is a pouched sheath (10A) having an opening (11) sized for finger insertion and other sides sealed. Both sides of said sheath (10) are allowed to be used.

Said pouched sheath (10A) can be formed by warp knitting machine, or made by superimposing two symmetrical fabrics (13, 14), leaving the opening (11) and sealing the remaining edges.

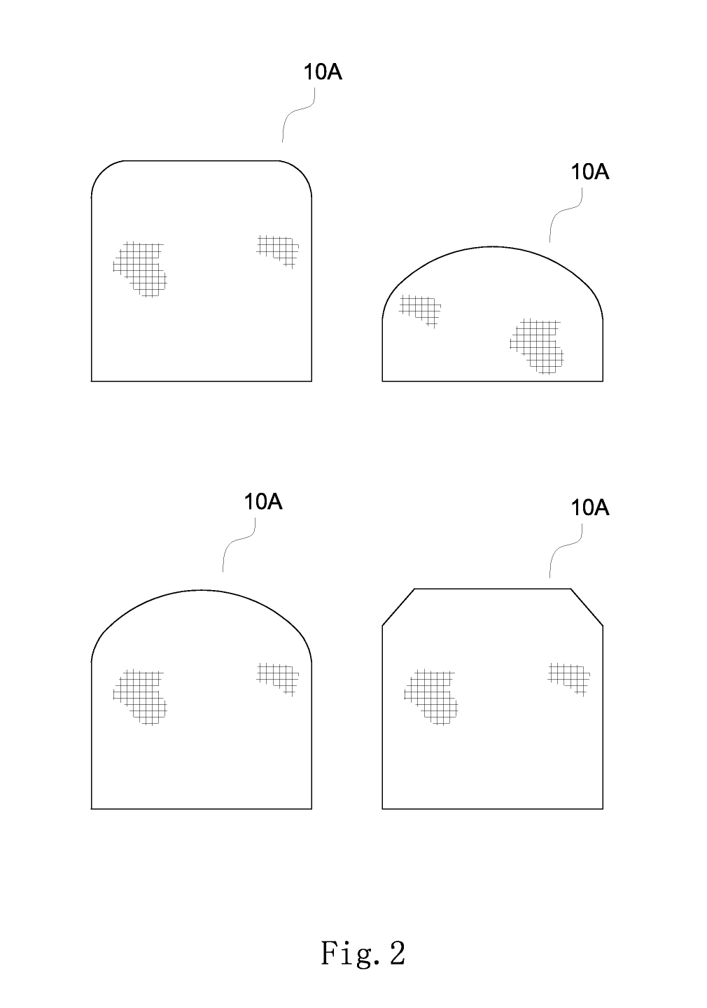

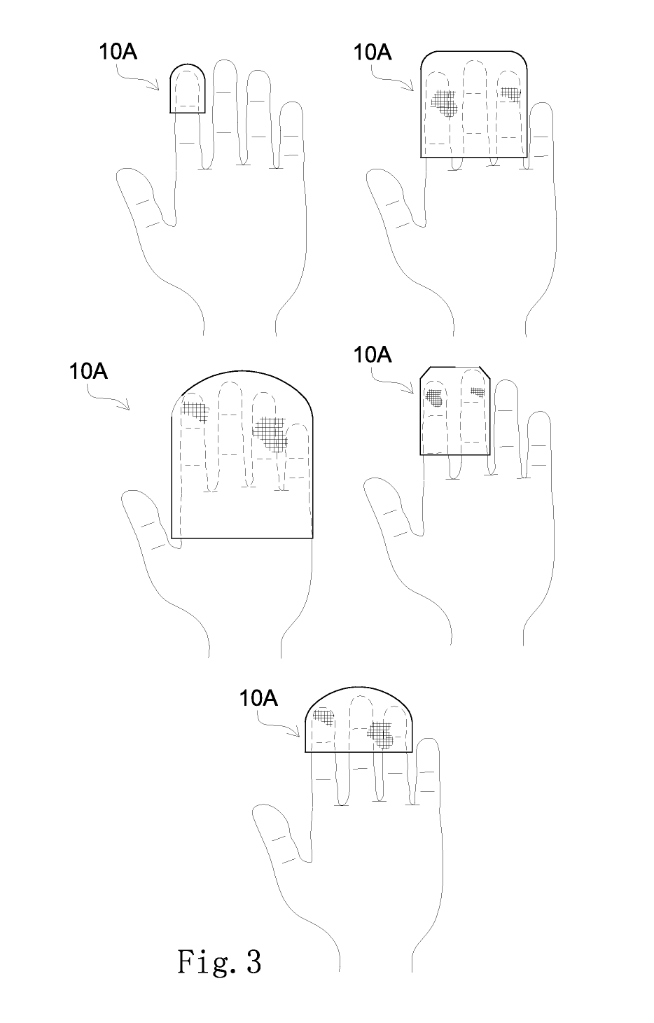

Alternative shapes of said pouched sheath (10A) are illustrated in FIG. 2, which the shape can be a rectangular, a semicircular, an oval, or a polygon shape. Applicable usages for wearing the pouched sheath (10A) are shown in FIG. 3, with openings (11) varied to correspond to the number of fingers to be inserted. The width of the opening (11) is recommended to be sized for allowing insertion of three to four fingers. The length of said pouched sheath (10A) vary, with a length extending from the fingertip to at least the first joint of any finger, or from the fingertip extending to the palm, depending upon the number of joints to be covered. The preferred length would be five to eight centimeters so that the coverage may also extend to the second joint or even the third for providing advantage that the inner portion of fingers will not come into contact with the skin.

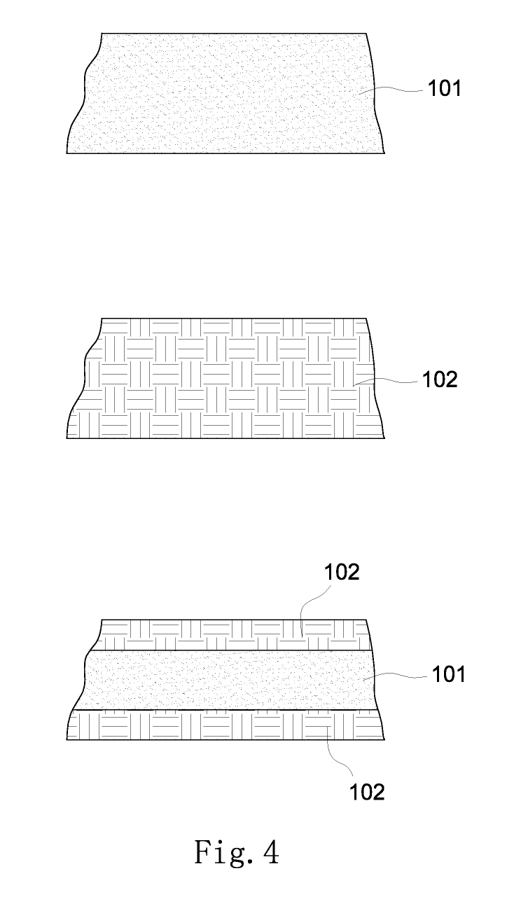

Furthermore, the pouched sheath (10A) is made of an absorber which is a material owns water- and/or oil-absorbing properties. As shown in FIG. 4, the absorber can comprises of a single cotton layer (101), a single non-woven fabric layer (102), or a composite structure including a cotton layer (101) as an inner layer and a non-woven fabric (102) as an outer layer. Said non-woven fabric layer (102) is made of fiber and porous fiber. The main function of the absorber is to absorb water-, oil-, milk-, cream- or powder-based materials, the possible applications thereof are described as follow:
1. Applying cosmetics: referring to third figure of FIG. 4, when the absorber of said pouched sheath (10A) has said composite structure, it may be wetted with water or toner for applying cosmetic.
2. Cleansing and exfoliation: referring to second figure of FIG. 4, when the absorber of said pouched sheath (10A) has a single non-woven fabric layer (102), made of either pulp fiber or artificial fiber or preferably the natural plant fiber; it provides a well defoliating effect and improves metabolism when used with toner.
3. Moisture replenishment: referring to first figure of FIG. 4, when the absorber of the pouched sheath (10A) has a single layer of natural cotton layer (101). It possesses strong absorbency and water retention capability, suitable for skin moisturizing toner to improve skin textures, ensuring a softer and more natural look.
4. Facial Mask: when said absorber made of natural cotton (101) is provided with a larger and thicker (actual thickness 0.3 mm.) size, it may be used as facial mask owing to its strong water absorbability.

Furthermore, the absorbent layer of the finger sheath (10) can be made of fiber, porous fiber, or a combination of both. The pores provide hydrophilicity and skin-friendliness which gives a cool feeling to skin when used with pure water. Besides, increased porosity improves air permeability and moisture absorption. Furthermore, elasticity is also improved for a softer surface quality.

Said sheath (10A) made of pure cotton (101), may also be coated with medical cream, ointment, or disinfectant, and applied to skin or wounds. It is best used as a medical tool for cleansing and many other purposes, and therefore may be used in place of the traditional cotton pads.

The sheath (10A) made of non-woven fabric layer (102) can also be used to polish shoes when dampened with water. It is one of the best tools used for polishing leather shoes. If said layer (102) is made of crude fiber, then it may be used either for defoliation or bath descaling.

The sheath (10A) made of said composite material (101, 102) can be used for acupressure massage and hydraulic massage when used with massaging cream or oil.

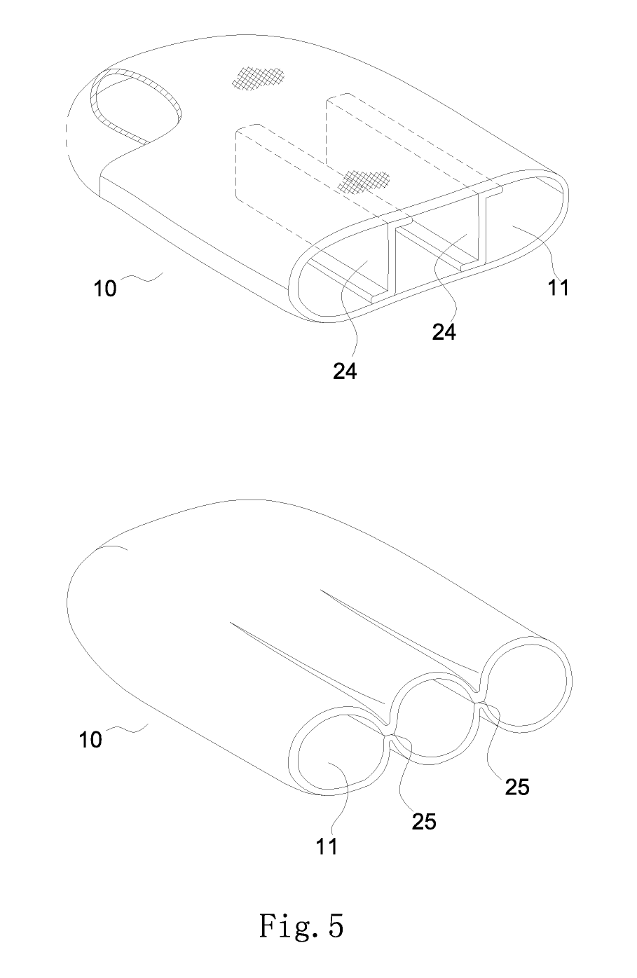

FIG. 5 illustrates two alternative applications of the first embodiment. The inner portion of sheath (10A) is provided with pluralities of spacers (24) or separators (25). Said spacer (24) or separator (25) may either be sewn onto the sheath or molded onto it. The spacer (24) and separator (25) divide the opening (11) into pluralities sub openings to fit each finger respectively.

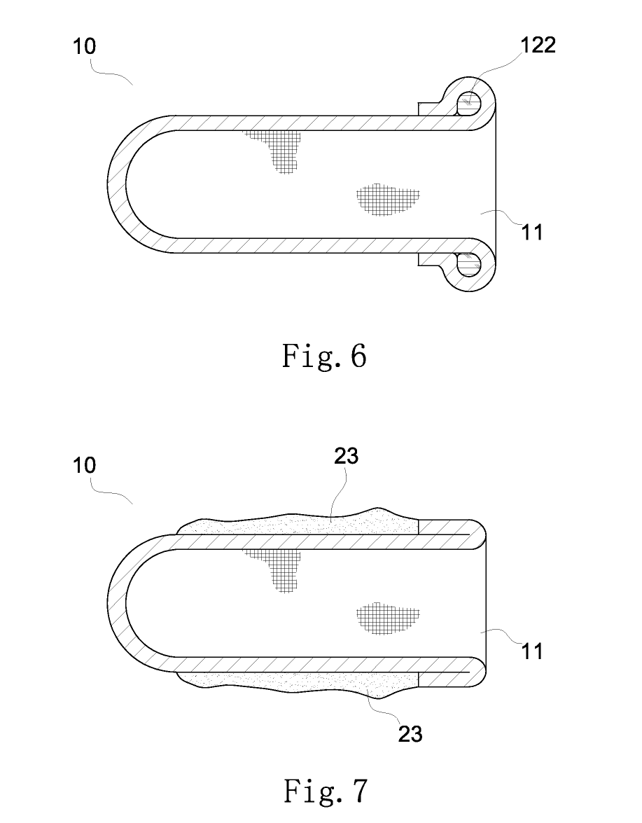

Referring to FIG. 6, a circular band (22) is provided about the circumference of the opening (11), forming a flexible and contracting edge to prevent the fingers slipping from said finger sheath (10).

As shown in FIG. 7, the surface of said finger sheath (10) is covered with a water-soluble layer (23) which is made of liquid-, milk-, cream-, gel, or powder based material. For example, if said layer (23) is made of facial lotion, thus the bubbles are generated when dampened with water may be used to cleanse the skin. it may also be used for skin care, scrubbing, slathering, or massage. To prevent dampness, the pouched sheath (10A) is preferably stored in a vacuum package. Said layer (23) can be provided in liquid-, cream-, gel, or powder state for stronger adhesive effect.

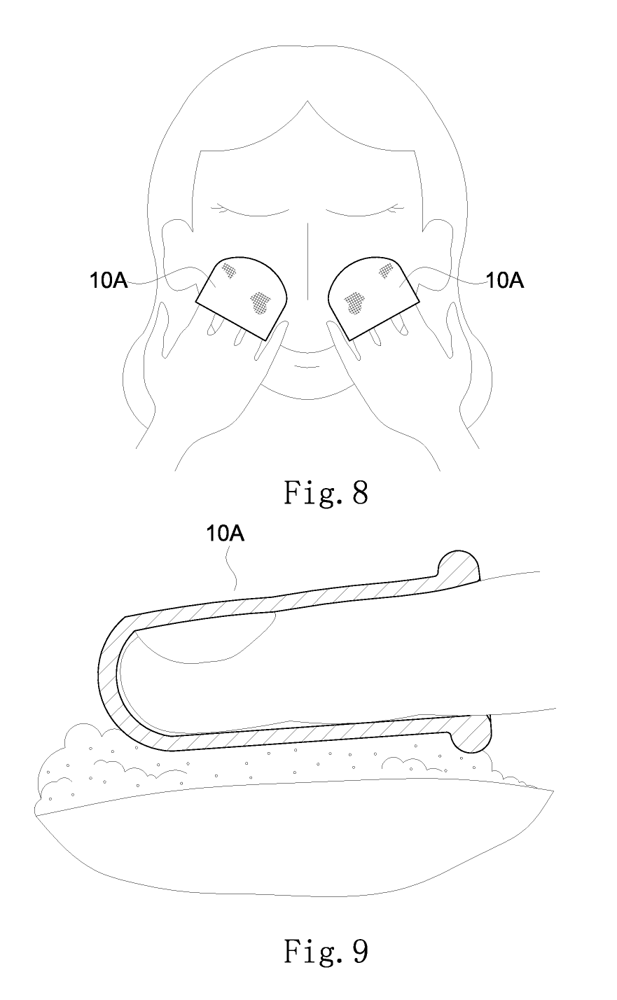

FIGS. 8 and 9 shows that when coated or smeared with cleansing lotion or detergent, said pouched sheath (10A) may be used for defoliation, make-up removal, and dirt-removal. It may also be used as a skin care-product when used with facial lotion, hand lotion, or the like. It may also be used for acupressure massage and hydraulic massage when coated with essential oil, and massage-milk or -cream. It may also be used for disinfection or cleaning wounds when medical lotion or cream is applied to it. Lastly, it may be used as a disposable cleansing product, skin care product, skin protection product, massage or medical product.

### Second Embodiment

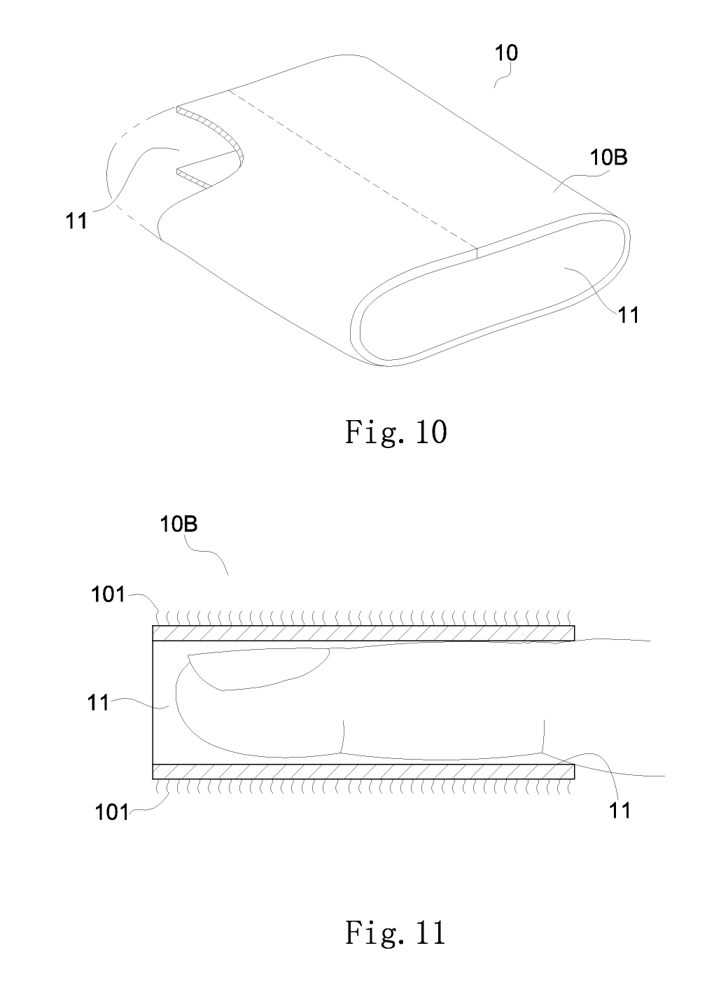

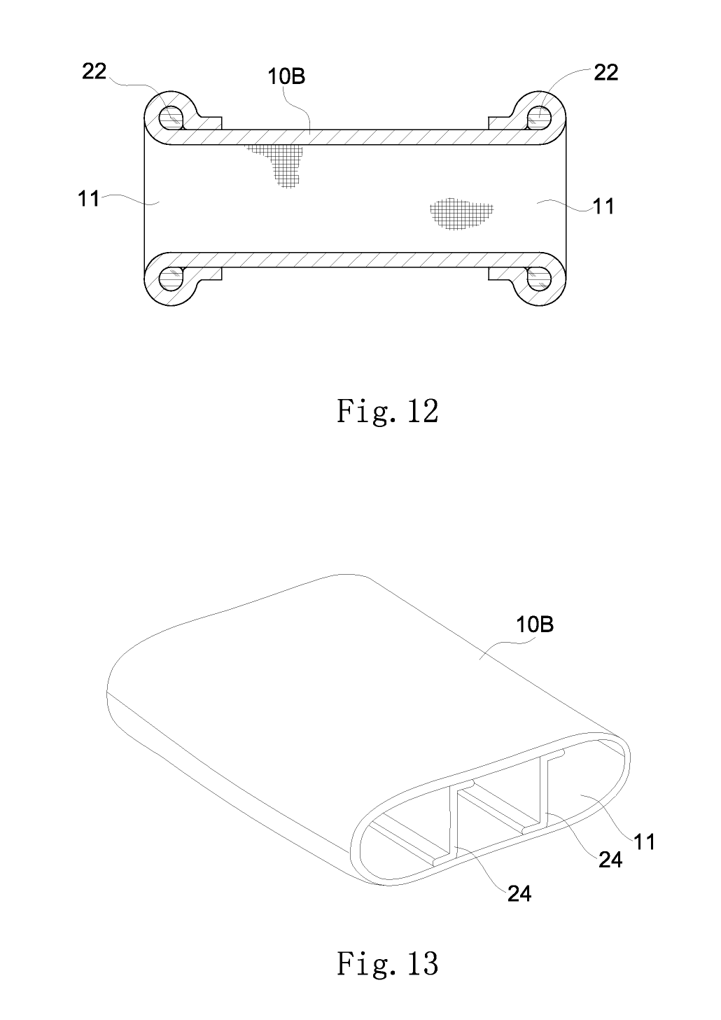

FIG. 10 shows the three-dimensional diagram of the second embodiment. FIG. 11 shows its elevation and schematic drawings, FIG. 12 another profile, and FIG. 13 another example of the three-dimensional diagram.

Referring to FIGS. 10-13, the finger sheath (10) can also be manufactured as a tubular sheath (10B) having two openings (11) set on both ends, and both faces may be used for cleansing, skin care, an application of cosmetic and medicinal ointment. Furthermore, both openings (11) of the tubular sheath (10B) are further attached with a circular band (22) respectively to form a flexible and contracting edge. Pluralities of spacers (24) or spacing threads (not shown) can be provided further to separate fingers.

The advantage of said sheath (10B) is allowed the insertion of fingers from both ends thereof.

### Third Embodiment

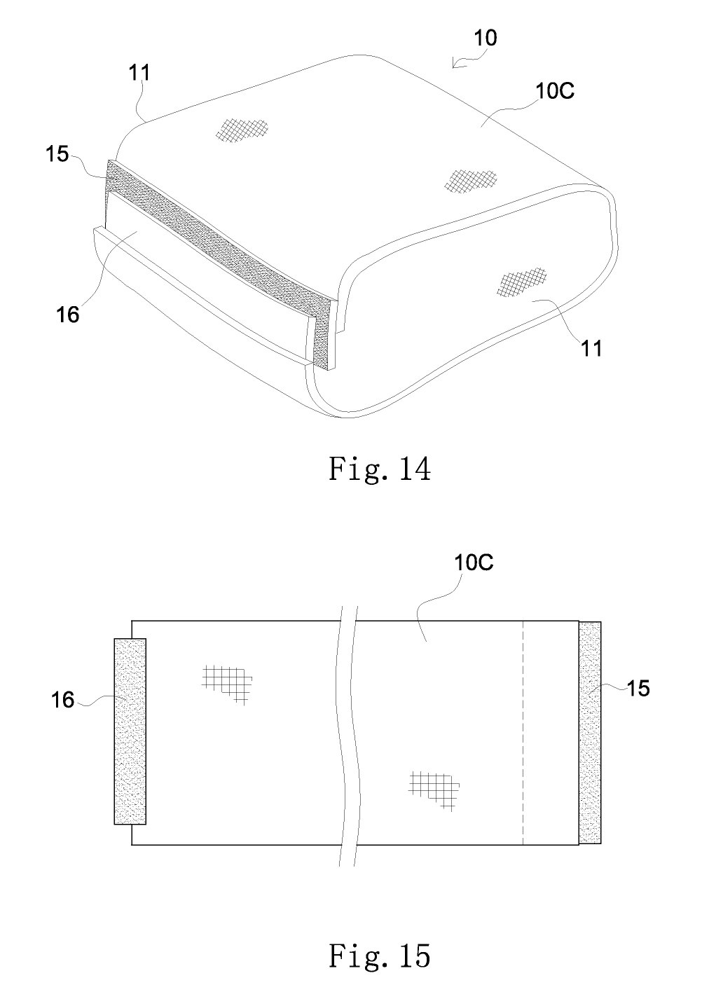

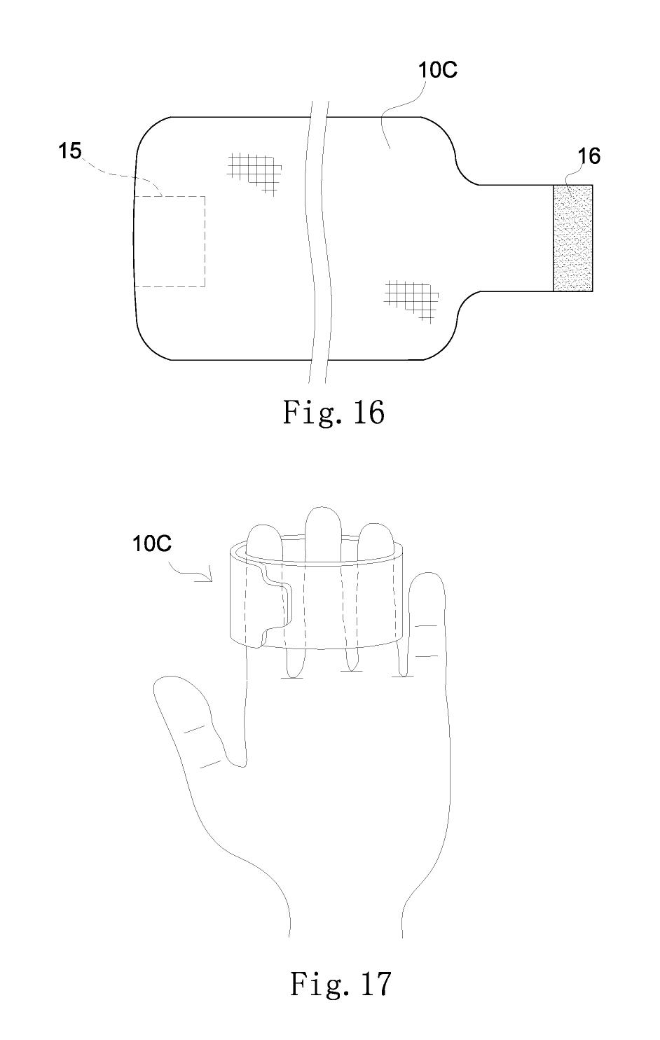

The third embodiment of the present invention is disclosed according to FIGS. 14-17. The finger sheath (10) is a band (10C) wrapped around fingers with both its ends sewn with a loop and a hook fastening tape (15, 16) that allow the length and tightness of the band to be adjusted.

### Fourth Embodiment

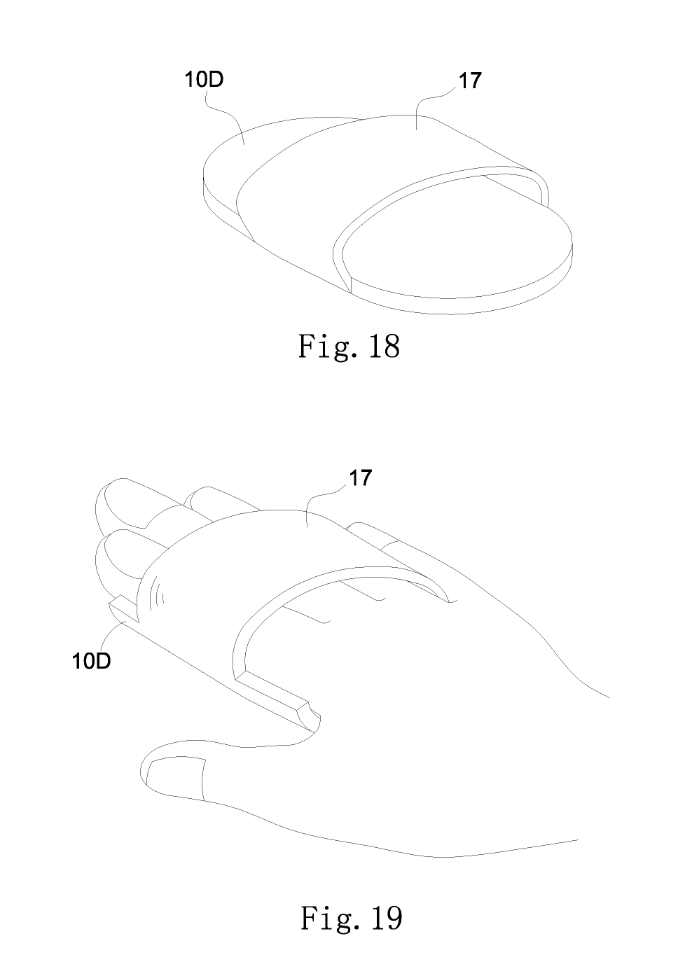

As shown in FIGS. 17-19, the forth embodiment discloses the finger sheath (10) manufactured as a plate-like draper (10D) having two lateral sides attach to an end of a bandage (17) respectively. Said draper (10D) may have a shape selected from rectangular, semicircular, oval, or hexagonal and said bandage (17) can be selected from a ribbon or an elastic drawstring

### Fifth Embodiment

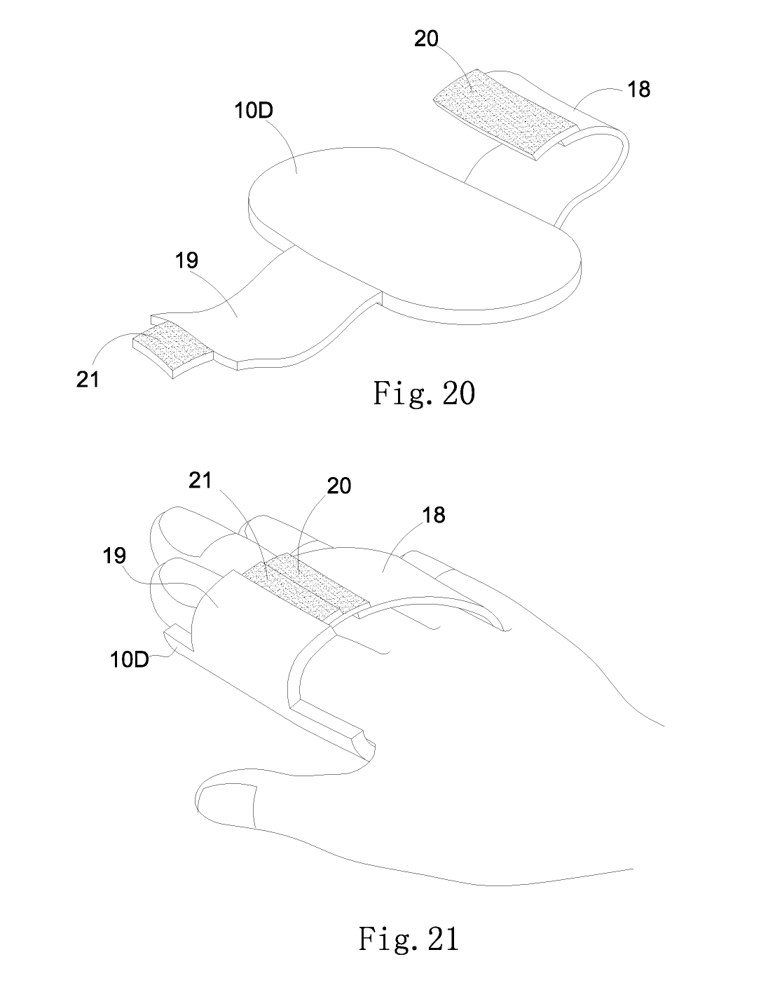

The fifth embodiment, as shown in FIGS. 20 and 21, discloses a finger sheath (10) as a plate-like draper (10D) having two lateral sides attached to an end of stripes (18, 19); and another end thereof attaches to a hook (20) or a loop portion (21) of an hook and loop fastener respectively. Said stripes (18, 19) can be selected from a ribbon or an elastic drawstring.

The present invention has been described via the detailed illustration of the preferred embodiments. Those skilled in the art can derive variations from the preferred embodiments without departing from the scope of the present invention. Therefore, the preferred embodiments shall not limit the scope of the present invention defined in the claims.

1. A finger sheath which covers at least one finger for cleansing, skin care, scrubbing, smearing, massage, and medical use is characterized in that:
the finger sheath (10) having at least one opening (11) covers a single or a plurality of finger, and having a length in a range from fingertip to first knuckle of the finger or from fingertip to palm; said sheath (10) is made of material with water absorbing and oil absorbing properties.

2. The finger sheath of claim 1, wherein said finger sheath (10) is a pouched sheath (10A) having one opening (11) set on a side thereof and others edges sealed; and both faces of said sheath (10) are useable.

3. The finger sheath of claim 1, wherein said finger sheath (10) is a tubular sheath (10B) having an opening (11) set respectively on both sides thereof for fingers insertion; and both faces of said sheath (10) are useable.

4. The finger sheath of claim 2, wherein the finger sheath (10) is made by superimposing two symmetrical fabrics (13, 14) and sealing others edges to have the opening (11) set on a side thereof.

5. The finger sheath of claim 3, wherein the finger sheath (10) is made by superimposing two symmetrical fabrics (13, 14) and sealing others edges to have the opening (11) set on a side thereof.

6. The finger sheath of claim 1, wherein the finger sheath (10) is a band (10C) having both ends thereof attach respectively to a hook (15) and a loop portion (16) of a hook and loop fastener.

7. The finger sheath of claim 1, wherein the finger sheath (10) is a plate-like draper (10D) having two lateral sides attach to an end of a bandage (17) respectively; said bandage (17) is selected from a ribbon or an elastic drawstring.

8. The finger sheath of claim 1, wherein the finger sheath (10) is a plate-like draper (10D) having two lateral sides attach to an end of a stripe (18, 19) respectively; and another end of said stripes attaches to a hook (20) or a loop (21) portion of an hook and loop fastener respectively; said stripes (18, 19) are selected from ribbons or elastic drawstrings.

9. The finger sheath of claim 1, wherein each of said opening (11) is encircled by an elastic band (22).

10. The finger sheath of claim 1, wherein a water-soluble layer (23) is provided onto said finger sheath (10).

11. The finger sheath of claim 1, wherein the material for manufacturing said finger sheath (10) is selected from a cotton layer (101), a non-woven fabric layer (102), or a composition having said cotton layer (101) as an inner layer and said non-woven fabric as an outer layer (102).

12. The finger sheath of claim 1, wherein a fiber, a porous fiber, or a mixture of both are selected for providing on a surface of the material.

13. The finger sheath of claim 2, a spacer (24) and a separator (25) are provided in said finger sheath (10) for spacing the fingers.

14. The finger sheath of claim 2, a spacer (24) and a separator (25) are provided in said finger sheath (10) for spacing the fingers.
